(19) United States
(12) Patent Application Publication
(10) Pub. No.: US 2024/0203592 A1
(43) Pub. Date: Jun. 20, 2024

(54) CONTENT PROVIDING METHOD, SYSTEM AND COMPUTER PROGRAM FOR PERFORMING ADAPTABLE DIAGNOSIS AND TREATMENT FOR MENTAL HEALTH

(71) Applicant: HAII CORP, Seoul (KR)

(72) Inventors: Hun Yeop JEONG, Gyeonggi-do (KR); Chan Mi PARK, Seoul (KR)

(73) Assignee: HAII CORP, Seoul (KR)

(21) Appl. No.: 18/537,439

(22) Filed: Dec. 12, 2023

(30) Foreign Application Priority Data
Dec. 15, 2022 (KR) 10-2022-0175549

(51) Int. Cl. G16H 50/20; G16H 10/20; G16H 20/70

(52) U.S. Cl. CPC G16H 50/20; G16H 10/20; G16H 20/70

(57) ABSTRACT

Proposed are a content providing method, system, and computer program for performing adaptable diagnosis and treatment for mental health. Provided is a content providing method for performing adaptable diagnosis and treatment for mental health, the method including a step in which a mental health condition of a user who uses a user terminal is classified through analysis of first digital biomarker data, a step in which mental health condition variation of the user who uses the user terminal is determined through analysis of second digital biomarker data, and a step in which content is changed and transmitted to the user terminal in accordance with the mental health condition variation.

### CROSS REFERENCE TO RELATED APPLICATION

The present application claims priority to Korean Patent Applications No. 1.0-2022-0175599, filed Dec. 15, 2022 the entire contents of which are incorporated herein for all purposes by this reference.

### BACKGROUND

### Technical Field

The present disclosure relates to a content providing method, system, and a computer program for performing adaptable diagnosis and treatment for mental health. In more detail, the present disclosure relates to a content providing method, system and computer program for performing adaptable diagnosis and treatment for mental health, the method, system, and computer program maintaining or changing an existing mental health diagnosis and treatment method, depending on whether mental health becomes better or worse while periodically diagnosing mental health through a digital biomarker and performing treatment in accordance with the diagnosis result.

### Description of the Related Art

Most modern people including not only workers who suffer from stress due to overwork but also students who are driven to severe competition and the elderly who are separated from their families and feel depressed have large and small problems in health care. In particular, as the Covid-9 pandemic has limited economic activities and normal life in recent years, the cases of metal disorders such as depression or anxiety disorders has greatly increased. Accordingly, efforts such as enhancing promotion of mental health services for supporting the citizens who suffer from mental disorders and receiving support from professional organizations to manage mental health of workers and students at companies and schools too are being made at the national level.

However, people still have a tendency to be unwilling to visit professional organizations such as a hospital to consult about mental health, and even though they visit professional organizations, they do not frankly answer about their mental health conditions in many cases. Accordingly, people cannot receive consultation about their exact mental health and cannot receive treatment at an appropriate period, so there are many cases of making the condition worse. Further, medicines are prescribed and cognitive behavioral therapy is performed for treatment after diagnosis of mental health, but using medicines for treatment may cause side effects such as sleepiness, memory impairment, withdrawal symptoms, and drug dependence.

Recently, mental health consultation is performed in a contactless manner through digital devices in some cases, but such consultation is performed only through questions, so the exactness is low and appropriate contactless treatment corresponding to diagnosis results is not made. Further, since following treatment after consultation about mental health is a one-off event, it is difficult to appropriately cope with variation of condition accompanying treatment. That is, when a condition depending on treatment is fed back and the condition becomes better or worse, following treatment should be adaptively changed, but it is difficult to achieve adaptable diagnosis and treatment through the existing contactless manner.

### SUMMARY

Accordingly, an objective of the present disclosure is to provide a content providing method, system, and computer program for performing adaptable diagnosis and treatment for mental health, the method, system, and computer program maintaining or changing an existing mental health diagnosis and treatment method, depending on whether mental health becomes better or worse while periodically diagnosing mental health through a digital biomarker and performing treatment in accordance with the diagnosis result.

In order to achieve the objectives described above, a content providing method for performing adaptable diagnosis and treatment for mental health of the present disclosure includes: a step in which first digital biomarker data obtained through a first application installed in a user terminal are received from the user terminal; a step in which a mental health condition of a user who uses the user terminal is classified through analysis of the first digital biomarker data; a step in which content corresponding to the mental health condition is transmitted to the user terminal to be provided to the user through a second application installed in the user terminal; a step in which second digital biomarker data obtained through the second application are received from the user terminal; a step in which mental health condition variation of the user who uses the user terminal is determined through analysis of the second digital biomarker data; and a step in which the content is changed and transmitted to the user terminal in accordance with the mental health condition variation.

The content providing method for performing adaptable diagnosis and treatment for mental health of the present disclosure may further include a step in which when homeostasis, which is property of a human body trying to maintain an internal environment at a constant level becomes a critical level or higher, it is determined that there is mental health condition variation and customized test information excluding items having constant homeostasis in standard test information transmitted to the user terminal in accordance with a preset first period is created and transmitted to the user terminal in accordance with a second period set shorter than the first period.

That is, the content providing method for performing adaptable diagnosis and treatment for mental health of the present disclosure may further include: a step in which standard test information is transmitted to the user terminal in accordance with a preset first period such that a standard test for classifying the mental health condition is provided through the first application installed in the user terminal; and a step in which answer data to the standard test are obtained through the first application and received from the user terminal,

The step in which the mental health condition of the user who uses the user terminal is classified may be a step in which the answer data are analyzed together with the first digital biomarker data, whereby the mental health condition of the user who uses the user terminal is classified.

Further, the step in which mental health condition variation is determined may be a step of determining that there is the mental health condition variation when homeostasis becomes a critical level or higher by analyzing the second digital biomarker data.

The method may further include a step in which when it is determined that there is the mental health condition variation, customized test information excluding items having constant homeostasis in the standard test information is created and transmitted to the user terminal.

The method may further include a step in which when it is determined that the mental health condition variation exists, the customized test information excluding items having constant homeostasis is repeatedly created and transmitted to the user terminal at every second period set shorter than the first period.

The standard test information and the customized test information may be any one of mental disorder question information that is provided to analyze a mental disorder of the user and cognitive function test information that is provided to classify a stage of dementia of the user.

The first digital biomarker data may include at least one or more items of information of Heart Rate Variability (HRV) information of the user, gaze information of the user, voice-related information of the user, and response time information of the user.

The heart rate variability information of the user may be extracted in a non-contact type by estimating a heart response by analyzing a color change from a real-time face image.

The gaze information of the user may be extracted by analyzing variation of a user's gaze from an image including user's eyes taken when the user's gaze follows a moving object.

The content may be any one of treatment content for treating a mental disorder of the user and a training content for training a cognitive function of the user.

The second digital biomarker data may be any one of voice-related information of the user and response information about at least one test of an arithmetic ability test, a memory test, and a gaze test.

The content providing method for performing adaptable diagnosis and treatment for mental health of the present disclosure may further include: receiving log data of each of the first application and the second application; determining whether any one application of the first application and the second application has reached any one value of a preset number of times of use and a preset use time with reference to the log data; and creating alarm information about use of an application that has not reached any one value of the preset number of times and the preset use time and transmitting the alarm information to the user terminal.

Meanwhile, a computer program stored in a computer-readable storage medium performs steps for performing adaptable diagnosis and treatment for mental health on the basis of digital biomarker data when the computer program is executed by at least one process of a device, wherein the steps include: a step in which first digital biomarker data obtained through a first application installed in a user terminal are received from the user terminal; a step in which a mental health condition of a user who uses the user terminal is classified through analysis of the first digital biomarker data; a step in which content corresponding to the mental health condition is transmitted to the user terminal to be provided to the user through a second application installed in the user terminal; a step in which second digital biomarker data obtained through the second application are received from the user terminal; a step in which mental health condition variation of the user who uses the user terminal is determined through analysis of the second digital biomarker data; and a step in which the content is changed and transmitted to the user terminal in accordance with the mental health condition variation.

A mental health management system that provides content for performing adaptable diagnosis and treatment for mental health on the basis of digital biomarker data includes: a user terminal configured to obtain first digital biomarker data by executing a first application and obtain second digital biomarker data by executing a second application; and an agent device configured to classify a mental health condition of a user who uses the user terminal by analyzing the first digital biomarker data transmitted from the user terminal, transmit content corresponding to the mental health condition to the user terminal, and determine mental health condition variation of the user who uses the user terminal by analyzing the second digital biomarker transmitted from the user terminal, wherein the agent device changes and transmits the content to the user terminal in accordance with the mental health condition variation.

According to the content providing method, system, and computer program for performing adaptable diagnosis and treatment for mental health, since mental health is diagnosed and treated in a digital manner, there is no danger of side effects and it is possible to receive diagnosis and treatment for mental health without limitation in time and space as long as a terminal is given.

Since a change in thinking of an individual is induced through Cognitive Behavioral Therapy (CBT) that is applied as a treatment method, it is possible to correct cognitive distortion, reduce maladaptive behaviors through intervention in behavior correction, and finally attenuate anxiety.

Further, by periodically checking whether a mental health condition becomes better or worse in accordance with treatment, it is possible to perform adaptable management that maintains or changes existing diagnosis and treatment for mental health.

Further, since tests are performed only for the items having condition variation, it is possible to reduce time for tests and the fatigue of a user responding to tests. Further, since only tests concentrating on items having mental health condition variations are performed, it is possible to deeply analyze relevant symptoms and deeply determine change of the relevant symptoms.

### BRIEF DESCRIPTION OF THE DRAWINGS

The above and other objectives, features and other advantages of the present disclosure will be more clearly understood from the following detailed description when taken in conjunction with the accompanying drawings, in which:

FIG. 1 is a view simply showing the configuration of a mental health management system according to all embodiments of the present disclosure;

FIG. 2 is a block diagram showing the configurations of an agent device and a user terminal that constitute the mental health management system according to all embodiments of the present disclosure;

FIGS. 3A to 3C are views illustrating a method of diagnosing a mental disorder through a first application in accordance with a first embodiment of the present disclosure;

FIGS. 4A to 4C are views illustrating a method of treating a mental disorder through a second application in accordance with the first embodiment of the present disclosure;

FIGS. 5A to 5C are views illustrating a method of diagnosing cognitive functions through the first application in accordance with a second embodiment of the present disclosure;

FIGS. 6A to 6C are views illustrating a method of training cognitive functions through the second application in accordance with the second embodiment of the present disclosure; and

FIG. 7 is a flowchart illustrating a content providing method of the mental health management system according to all embodiments of the present disclosure.

### DETAILED DESCRIPTION

Hereafter, a method of performing adaptable diagnosis and treatment for mental health according to a preferred embodiment of the present disclosure and a mental health management system for performing the method are described in detail with reference to the accompanying drawings.

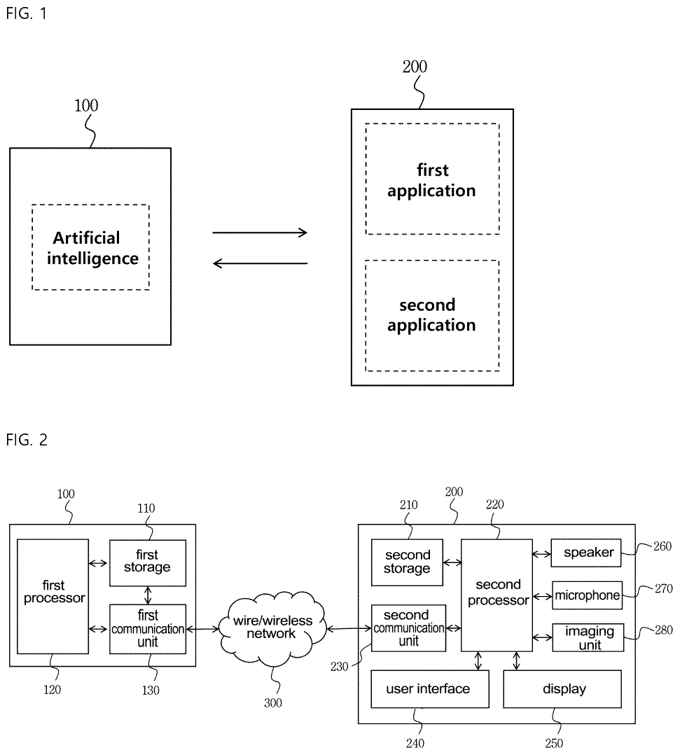

FIG. 1 is a view simply showing the configuration of a mental health management system according to all embodiments of the present disclosure and FIG. 2 is a block diagram showing the configurations of an agent device and a user terminal that constitute the mental health management system according to all embodiments of the present disclosure.

First, referring to FIG. 1, a mental health management system of the present disclosure includes an agent device 100 that adaptively performs mental health diagnosis and treatment and a user terminal 200 in which a first application and a second application for mental health diagnosis and treatment are executed.

The agent device 100 may be any device that can transmit or receive data, content, services, applications, etc. The agent device 100 means a server in the present disclosure, but is not limited thereto and may mean a fixed-type device such as a Personal Computer (PC) or a mobile device such as a smartphone, a tablet PC, and a laptop, and may be used as a meaning that includes all of them unless specifically stated.

In this case, the server means a device or a system that transmits or receives data to or from various types of user terminals, that is, clients. The server, for example, may include a web server that provides web pages, web content, or web services, an advertising server that provides advertising data, a content server that provides content, an SNS server that provides a Social Media Service (SNS), a service server that is provided by manufacturing companies, a Multichannel Video Programming Distributor (MVPD) for providing Video on Demand (VoD) or streaming services, a service server that provides a pay service, etc., etc.

Meanwhile, the user terminal 200 of the present disclosure is connected with the agent device 100 and can transmit, receive, and display predetermined data. The user terminal 200 may be implemented as a mobile terminal such as a mobile phone, a smartphone, a tablet PC, and Ultrabook. However, the user terminal is not limited thereto and may include fixed-type devices too such as a Personal Computer (PC), a microprocessor, a mainframe computer, a digital processor, and a device controller.

Test information for classifying the mental health condition of a user is provided and first digital biomarker data about the user and answer data to the test can be obtained through the first application that is executed in the user terminal 200. A mental health condition means a condition in which the person can always independently and autonomously handle normal life, has resistance against diseases, and can smoothly manage individual and social life, that is, a mentally mature condition. Mental health conditions related to mental disorders or cognitive functions are included in the mental health condition stated in the present disclosure, and test information for analyzing mental disorders or test information for analyzing cognitive functions is provided through the first application. The first digital biomarker data and answer data obtained through the first application are transmitted to the agent device 100, and the agent device 100 classifies the mental health condition of the user by analyzing the first digital biomarker data and answer data.

Content corresponding to the mental health condition of a user is provided and second digital biomarker data are obtained through the second application that is executed in the user terminal 200. That is, through the second application, content for mental disorder treatment or cognitive function training is provided to a user and second digital biomarker data corresponding to feedback for the content from the user are obtained. The second digital biomarker data are transmitted to the agent device 100 and the agent device 100 determines mental health condition variation of the user by analyzing the second digital biomarker data.

Hereafter, the entire configuration and operation of the agent device 100 and the user terminal 200 are described in more detail with reference to FIG. 2.

The agent device 100 and the user terminal 200 are connected to each other through a wire/wireless network 300 and transmit and receive data to and from each other. The wire/wireless network 300 is a common term of a communication network that supports various communication standards and protocols for pairing or data transmission and reception between the agent device 100 and the user terminal 200. The wire/wireless network 300 includes all of communication networks that are currently supported or will be supported later by standards, and can support one or all of communication protocols for the communication networks.

The agent device 100 of the present disclosure includes a first storage 110, a first processor 120, and a first communication unit 130. The components may be integrated, added, or omitted, depending on the actually implemented specifications of the agent device 100. That is, if necessary, two or more components may be combined into one component or one component may be divided into two or more components. Further, the functions that are performed in blocks, respectively, are provided to explain embodiments of the present disclosure, and the detailed operation or devices for them do not limit the range of the right of the present disclosure.

The first storage 110 can store data that support various functions of the agent device 100. That is, the first storage 110 can store many application programs that are driven in the agent device 100, and data, commands, and at least one or more program instructions for operating the agent device 100. At least some of the application programs may exist in the agent device 100 from the moment of delivery from a warehouse for the fundamental functions of the agent device 100 or may be downloaded from external servers through wireless communication.

In the present disclosure, the first application, test information for analyzing mental disorders or test information for analyzing cognitive functions that are provided through the first application, and first digital biomarker data and answer data to the test that are obtained through the first application are stored in the first storage 110. Further, the second application and second digital biomarker data that are obtained through the second application are stored in the first storage 110. The first application and the second application stored in the first storage 110 are transmitted to the user terminal 200 through the wire/wireless network 300 and installed therein. The first and second digital biomarker data and answer data stored in the first storage 110 are analyzed by an artificial intelligence model that is executed at the first processor 120 to be described below to classify the mental condition of the user and determine mental condition variation.

The first processor 120 can control general operation of the agent device 100 and can provide or process appropriate information or functions by processing signals, data, information, etc. that are input or output through the components of the agent device 100 or by driving the application programs stored in the first storage 110. Further, the first processor 120 can control at least some of the components of the agent device 100 to drive the application programs stored in the first storage 110. Further, the first processor 120 can operate, in combination, at least two or more of the components included in the agent device 100 to drive the application programs.

The first processor 120 may be composed of one or more cores and may be any processor of various common processors. For example, the first processor 120 may include a Central Processing Unit (CPU), a General Purpose Graphics Processing Unit (GPUGP), a Tensor Processing Unit (TPU), etc. of a device. Further, the processor 120 may be composed of dual processor or other multi-processor architectures.

In the present disclosure, the first processor 120 classifies a mental health condition by analyzing the first digital biomarker data and answer data to a test stored in the first storage 110 and performs control such that content for mental disorder treatment or cognitive function training is correspondingly transmitted to the user terminal 200. Further, the first processor 120 changes and controls the content for mental disorder treatment or cognitive function training be transmitted to the user terminal 200 in accordance with mental health condition variation by analyzing the second digital biomarker data stored in the first storage 110.

Describing in more detail, the first processor 120 analyzes first digital biomarker data and answer data obtained through the first application. The first application is executed in the user terminal 200 and a standard test for analyzing the mental health condition of a user is provided through the first application in accordance with a preset first period. The standard test stated herein is a test for analyzing mental disorders or cognitive functions and includes all items related to mental disorders or cognitive functions. A standard test about mental disorders includes all of depression, anxiety disorder, adjustment disorder, Posttraumatic Stress Disorder (PTSD), insomnia, and suicide that are representative six major mental disorders.

Standard test information for analyzing mental disorders or cognitive functions is provided to a user through the first application in accordance with a predetermined first period, and corresponding answer data are input to the user terminal 200. The first processor 120 analyzes the answer data and first digital biomarker data that are transmitted from the user terminal 200 and makes content for mental disorder treatment or cognitive function training in accordance with the analysis result be transmitted to the user terminal 200.

When classifying the answer data and first digital biomarker data, the first processor 120 classifies given data into classes using a classification model. The classification model is an Artificial Intelligence (AI) model that determines what kinds data pertain to, and, in the present disclosure, a stacking ensemble model created by combining various classification models is used. The stacking ensemble model is a method of performing estimation one time through an individual model and performing again estimation by dividing into the estimated result into learning data and test data, and is better in performance than a single model.

Further, the first processor 120 revises a customized treatment plan using a reinforcement learning model. That is, the first processor 120 estimates a later condition by applying a reinforcement learning model on the current mental health condition of a user classified through the stacking ensemble model, creates content suitable for treating or training the later condition, and makes the content be transmitted to the user terminal 200. In this case, the content for mental disorder treatment or cognitive function training is provided to the user through the second application that is executed in the user terminal 200.

When second digital biomarker data obtained through the second application are transmitted from the user terminal 200 and stored in the first storage 110, the processor 120 determines mental health condition variation by analyzing the second digital biomarker data. In this case, the second digital biomarker data are fed back the result of mental disorder treatment or cognitive function training and used to create new content for treatment or training. Steps in which new content is created in accordance with the result of determining the mental health condition and is transmitted to the user terminal 200 and new second digital biomarker data are obtained are periodically repeated. The first processor 120 can maintain high accuracy by analyzing second digital biomarker data that are periodically obtained using a stacked ensemble model.

Meanwhile, when determining that mental health condition variation exists, the first processor 120 creates and transmits customized test information to the user terminal 200. The first processor 120 determines that there is mental health condition variation when homeostasis is over a critical level as the result of analyzing the second digital biomarker data. That is, a human body has homeostasis that tries to maintain the internal environment at a constant level and has a tendency to try to maintain homeostasis also in terms of psychiatry. When such homeostasis becomes a preset critical level or higher, it is determined that there is mental health condition variation, and customized test information excluding items having constant homeostasis in standard test information is created and transmitted to the user terminal 200.

For example, a standard test for testing mental disorders includes at least one item of depression, anxiety disorder, adjustment disorder, Posttraumatic Stress Disorder (PTSD), insomnia, and suicide, and when it is determined that the homeostasis for the item of depression is constant, customized test information includes only tests for the other items except the test for determining the item of depression. Alternatively, when variation in homeostasis for anxiety disorder is sensed due to a change for the better of adjustment disorder, customized test information includes only a test for determining the item of adjustment disorder.

Alternatively, when detecting abnormality by analyzing second digital biomarker data that are periodically transmitted from the user terminal 200, the first processor 120 creates and transmits customized test information for checking only the item having the abnormality detected to the user terminal 200. For example, when abnormality is detected in anxiety disorder and insomnia as a standard test result for testing mental disorders, customized test information for checking only the matters related to anxiety disorder and insomnia is created and transmitted to the user terminal 200.

The standard test information is transmitted to the user terminal 200 in accordance with the preset first period and customized test information is transmitted to the user terminal 200 in accordance with a second period set shorter than the first period. New customized test information is created except items having constant homeostasis every time it is determined that mental health condition variation exists within the first period, and is transmitted to the user terminal in accordance with the second period. For example, when standard test information is transmitted to the user terminal 200 every three months for mental health analysis, new customized test information is created and transmitted to the user terminal 200 every one month. Since items having constant homeostasis are excluded from customized test information, that is, only items having variation is selected and included, it is possible to reduce time to response to tests and a user has only to answer only items having variation without responding to the same tests every time, so it is possible to reduce a mental burden accompanying response to tests.

Further, the first processor 120 may create and transmit alarm information to the user terminal 200 to provide a warning that advises a user to visit a hospital or consult professionals, if necessary, when a mental health condition becomes worse. For example, it is possible to create alarm information when abnormality is accumulated by analyzing second digital biomarker data that are obtained while mental health treatment or cognitive function training is periodically performed.

In addition, the first processor 120 can receive log data of each of the first application and the second application from the user terminal 200. The first processor 120 can recognize whether the first application or the second application has been used by a preset number of times of use and by a preset time on the basis of the log data. When determining that the first application or the second application has been used by the number of times of use smaller than a preset value or by time shorter than a preset value, the first processor 120 can transmit alarm information including a message for promoting use of the corresponding application to the user terminal 200. Accordingly, the user recognizes the alarm information and responds to the test that is provided through the first application by a preset number of times or uses content that is provided through the second application by preset time.

However, the present disclosure is not limited to the embodiment described above and a second processor 220 of the user terminal 200 to be described below may obtain log data of the first application and the second application and output alarm information corresponding to the log data to promote use of each of the first application and the second application.

The first communication unit 130 may include one or more modules enabling wire/wireless communication between the agent device 100 and the wire/wireless network 300, between the agent device 100 and another device, or between the agent device 100 and an external server. Further, the first communication unit 130 may include one or more modules that connect the agent device 100 to one or more networks. The first communication unit 130, which means a module for connection to the wire/wireless network 300, may be disposed inside or outside the agent device 100 and may be configured to transmit and receive wire/wireless signals.

In the present disclosure, the first communication unit 130 transmits the first and second applications to the user terminal 200 such that the applications are installed in the user terminal 200. Further, the first communication unit 130 transmits standard test information and customized test information, receives answer data and first digital biomarker data obtained through the first application, transmits content, and receives second digital biomarker data obtained through the second application.

Meanwhile, the user terminal 200 of the present disclosure can be paired or connected with the agent device 100 through the wire/wireless network 300, so the user terminal 200 can transmit, receive, and display predetermined data. The user terminal 200 includes a second storage 210, a second processor 220, a second communication unit 230, a user interface 240, a display 250, a speaker 260, a microphone 270, and an imaging unit 280. The components may be integrated, added, or omitted, depending on the actually implemented specifications of the user terminal 200. That is, if necessary, two or more components may be combined into one component or one component may be divided into two or more components. Further, the functions that are performed in blocks, respectively, are provided to explain embodiments of the present disclosure, and the detailed operation or devices thereof do not limit the right range of the present disclosure.

In the second storage 210, first and second applications downloaded from the agent device 100 are stored, and first and second test information transmitted from the agent device 100 and content for treatment or training are stored.

The second processor 220 performs the first application stored in the second storage 210 such that the first and second test information is displayed through the display 250, and creates answer data and performs control such that the answer data are transmitted to the agent device 100 in accordance with an instruction by a user that is input through the user interface 240. Further, the second processor 220 obtains first digital biomarker data by executing the first application and then performs control such that the first digital biomarker data are transmitted to the agent device 100.

The second processor 220 executes the second application stored in the second storage 210 such that content is displayed through the display 250 and a content-related sound is output through the speaker 260. Further, the second processor 220 obtains second digital biomarker data by executing the second application and then performs control such that the second digital biomarker data are transmitted to the agent device. In this case, the second digital biomarker data is feedback on content that is provided to a user for treatment or training, and is used to create new content.

The first and second digital biomarker data include at least one or more items of information of Heart Rate Variability (HRV) information of a user, gaze information of a user, voice-related information of a user, and response time information of a user. HRV information and gaze information of a user are extracted from a face image of the user taken through the imaging unit 280, and voice-related information of a user is extracted from the voice of the user that is input through the microphone 270. Response time information of a user, which is information that is provided through the display 250 or information about the response time to content by the user, is extracted by recognizing the speed of a touch or key input by the user that is input through the user interface 240.

The second communication unit 230 receives the first and second applications, the first and second test information, and the content from the agent device 100 by communicating with the agent device 100 through the wire/wireless network 300. Further, the second communication unit 230 transmits the first digital biomarker data and answer data obtained through the first application to the agent device 100 and transmits the second digital biomarker data obtained through the second application to the agent device 100.

The use interface 240 is implemented in a key type or in a touch type and receives instructions by a user. A user can input desired instructions through the user interface 240 to execute the first and second applications.

The display 250 displays information that is processed by the user terminal 200. For example, the display 250 can display execution image information of application programs that are driven in the user interface 200 or User Interface (UI) and Graphic User Interface (GUI) information according to this execution image information. The display 250 may be combined with a touch-type user interface 240 and may receive instructions by a user. The first and second applications are displayed on the display 250 of the present disclosure, whereby test information or content is provided to a user. The speaker 260 outputs audio data received through the second communication unit 230 or stored in the second storage 210. Further, the speaker 260 may output audio data related to functions that are performed by the user terminal 200. In the present disclosure, the speaker 260 outputs audio data related to execution of the first and second applications.

The microphone 270 converts external sound signals into audio data and the converted audio data can be used in various ways in accordance with the functions that are being performed by the user terminal 200. In the present disclosure, the microphone 270 converts the voice of a user reading content into audio data.

The imaging unit 280 may include one or a plurality of cameras. That is, the user terminal 200 includes one or a plurality of cameras on at least one surface of the front surface or the rear surface. The imaging unit 280 can process image frames of still images or videos that are obtained by an image sensor. The processed image frames can be displayed on the display 250 or stored in the second storage 210.

Meanwhile, the imaging unit 280 included in the user terminal 200 may be disposed such that a plurality of cameras forms a matrix structure. A plurality of items of image information having various angles or focuses can be input to the user terminal through the cameras forming a matrix structure, as described above. Further, the imaging unit 280 may include a plurality of lenses arranged in at least one line. The plurality of lenses may be arranged in a matrix form, which may be called an array camera. When the imaging unit 280 is an array camera, it is possible to take images in various ways using a plurality of lenses and it is possible to obtain images with high quality.

In the present disclosure, the imaging unit 280 can take an image of the face of a user and can obtain an image including movement of the user's eyes in linkage with displaying of moving objects on the display 250.

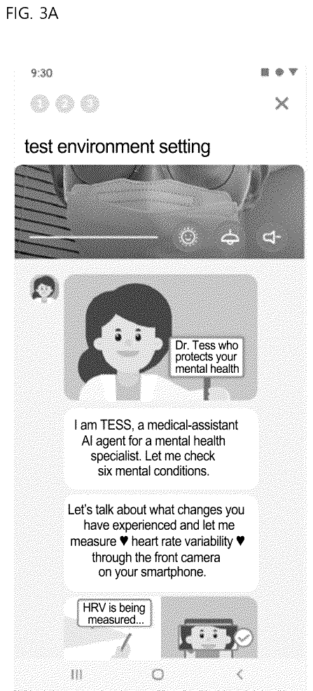

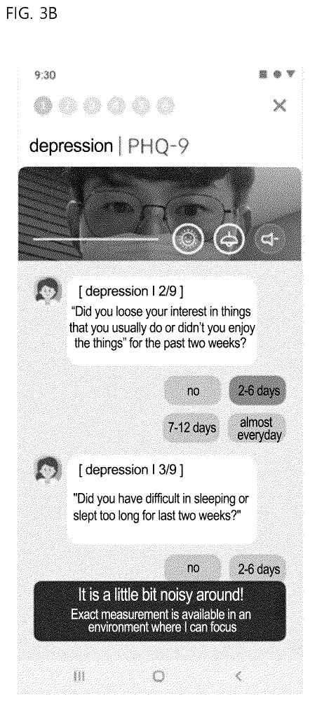

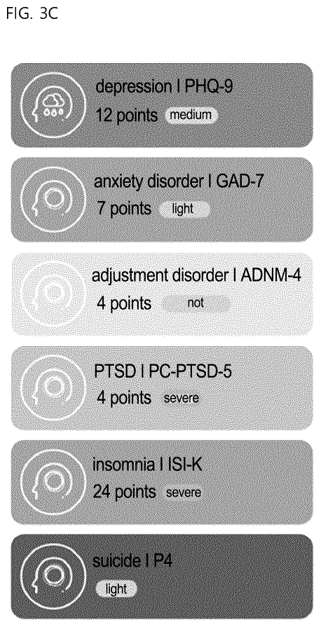

FIGS. 3A to 3C are views illustrating a method of diagnosing a mental disorder through a first application in accordance with a first embodiment of the present disclosure.

In the first embodiment of the present disclosure, the first application obtains HRV information as first digital biomarker data, provides mental disorder question information that is test information that is provided to analyze a mental disorder, and obtains answer data corresponding thereto.

Referring to FIG. 3A, HRV information is measured through the user terminal 200, transmitted to the agent device 100, and stored in the first storage 110 as first digital biomarker data. HRV information means variation of the interval between heartbeats and is a clinical representative index that can best measure six major mental disorders related to stress. Depression, anxiety disorder, adjustment disorder, Posttraumatic Stress Disorder (PTSD), insomnia, and suicide are included in the six major mental disorders. In the first embodiment of the present disclosure, the HRV information is extracted from the face image of a user taken through the imaging unit 280. That is, the HRV information is extracted in a non-contact type of estimating a heart response by analyzing a color change from a real-time face image. Describing in more detail, first, a face is detected from a taken image and then the face is defined, whereby variation of the face is traced. Further, fine movement signals of the face are extracted and then filtered, whereby HRV information is extracted.

Mental disorder question information that is provided to analyze depression of six major mental disorders is exemplified in FIG. 3B. When answers to question information about six major mental disorders are input through the user interface 240, the answer data are transmitted to the agent device 100 from the user terminal 200 and stored in the first storage 110. In this case, the question information that the user answers includes all of items for analyzing the conditions about the six major mental disorders, and standard question information is provided every three months and used to analyze the conditions about all of the six major mental disorders of the user.

A result of analyzing HRV information and answer data in relation to the six major mental disorders is exemplified in FIG. 3C. Of the six major mental disorders, depression was determined as ‘medium’, anxiety disorder and suicide were determined as ‘light’, adjustment disorder was determined as ‘not adjustment disorder’, and PTSD and insomnia were determined as ‘severe’. Treatment content for treating PTSD and insomnia determined as ‘severe’ of the determined mental health conditions is transmitted to the user terminal 200 from the agent device 100.

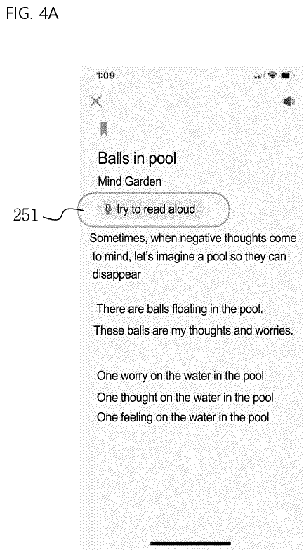

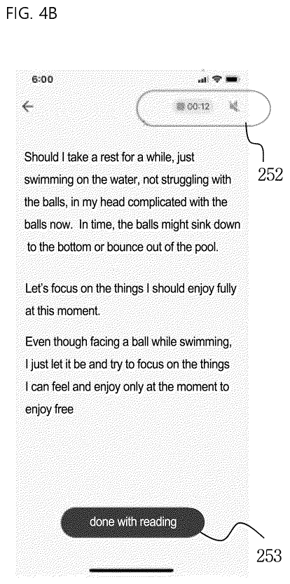

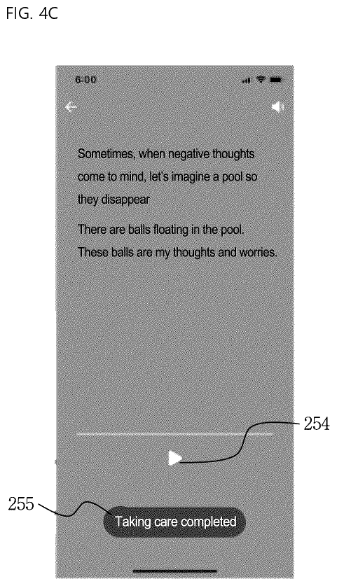

FIGS. 4A to 4C are views illustrating a method of treating a mental disorder through a second application in accordance with the first embodiment of the present disclosure.

Self-affirmation and Acceptance and Commitment Therapy (ACT) content using a self-talk technique is exemplified in FIGS. 4A to 4C. Self-affirmation and Acceptance and Commitment Therapy (ACT) is one of third generation of Cognitive Behavioral Therapy. Self-talk is a technique that is generally used to improve performance in sports, adjust anxiety or depression in an academic engagement clinic, etc. because there is an effect that the self-talk is useful in attention and emotion control. ACT is used to give help in self-reference activities so that a user can think with more intensive concentration on himself/herself in relation to his/her positive feelings and relevant experiences. On the other hands, ACT is used to concentrate on interaction of self-distancing that suppresses excessive self-absorption in incidents and experiences related to negative feelings.

As shown in FIG. 4A, when a user reads aloud treatment content that is provided when clicking a ‘try to read aloud’ button 251, a record indicator 252 that indicates ‘speaking out loud’ is turned on, as shown in FIG. 4B, and the user's voice is recorded. When the user finishes reading aloud and finishes recording by pressing a ‘done with reading’ button 253, the user's voice is stored in the second storage 210 and is transmitted and stored in the agent device 100 as second digital biomarker data. When the user presses a ‘replay’ button 254, the user can play and listen to his/her voice stored in the second storage 210, and then the user presses a ‘taking care completed’ button 255.

The user's voice transmitted to the agent device 100 is stored in the first storage 110 and the first processor 120 determines mental health condition variation by analyzing the user's voice stored in the first storage 110. When it is determined, for example, that the condition of PTSD changes from ‘severe’ to ‘light’ and the others are not changed as the result of analyzing the user's voice, the first processor 120 makes new treatment content, which is suitable for the changed condition, be provided to the user through the second application.

When the user is provided with the new treatment content and records his/her voice, the user's voice is transmitted to and analyzed in the agent device 100. While this process is repeated, the first processor 120 detects abnormality by continuously checking the user's voice. When homeostasis becomes a preset critical level, the first processor 120 detects that there is abnormality, and creates question information about PTSD having the abnormality detected, and provides the question information to the user in accordance with the period of one month. The user answers the question information about PTSD and the answer data are transmitted back to the agent device 100 and stored in the first storage 110. Since a user takes tests only for the items having condition variation, it is possible to reduce time for tests and the fatigue of a user responding to tests.

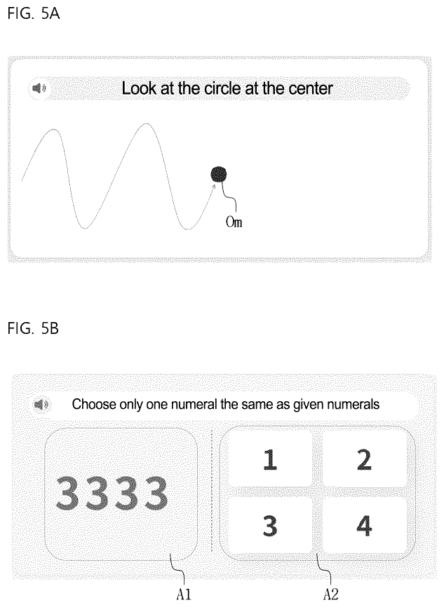

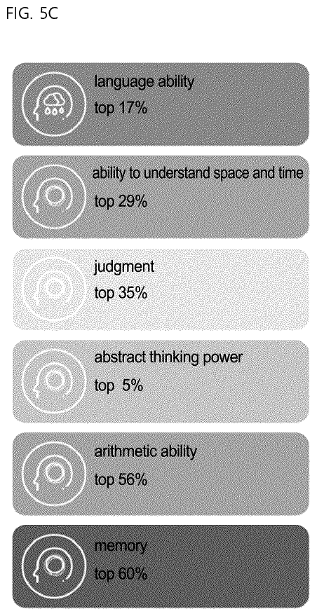

FIGS. 5A to 5C are views illustrating a method of diagnosing cognitive functions through the first application in accordance with a second embodiment of the present disclosure.

In the second embodiment of the present disclosure, the first application obtains user's gaze information as first digital biomarker data, provides cognitive function classification information that is test information that is provided to analyze a cognitive function, and obtains answer data corresponding thereto.

Referring to FIG. 5A, user's gaze information is obtained through the user terminal 200, transmitted to the agent device 100, and then stored in the first storage 110 as first digital biomarker data. That is, when a user's gaze follows a moving object Om displayed on the display 250, the second processor 220 controls the imaging unit 280 to obtain an image including the user's eyes. Further, the second processor 220 obtains user's gaze information by analyzing variation of the user's gaze from the image including the user's eyes.

In this case, the user's gaze information can be calculated using the coordinate values of the user's pupils analyzed from the image including the user's eyes. The coordinate value of a pupil may be the coordinate value of the point at which the center of the pupil is positioned and may be a coordinate value related to the edge of the pupil, but is not limited thereto. The user's gaze information may include at least one of accuracy information that is calculated on the basis of the movement distance of the user's eyes and the movement distance of a moving object, latency information that is calculated on the basis of the point in time at which a moving object starts to move and the point in time at which the user's eyes start to move, and speed information related to the movement speed of the user's eyes.

The accuracy information may be information about whether a user is accurately gazing at a moving object Om. In this case, the accuracy information may be determined using information about a movement distance of the user's gaze and information about a movement distance of the moving object Om. In detail, as the value obtained by dividing the movement distance of the user's gaze by the movement distance of the moving object Om comes close to 1, it is possible to recognize that the user is accurately gazing at the moving object Om.

The latency information may be information for checking a response speed of a user. That is, the latency information may include information of the time taken from the point in time at which a moving object Om starts to move and the point in time at which the user's eyes start to move.

The speed information may mean a movement speed of user's eyes. That is, the speed information can be calculated on the basis of information of the movement distance of user's eyes and information of the time taken by the user's eyes to move. However, the present disclosure is not limited thereto and the second processor 220 creates the position path of a user's gaze and obtains a speed value by differentiating the position path, thereby being able to calculate speed information.

As described above, user's gaze information including at least one or accuracy information, latency information, and speed information is transmitted to the agent device 100 and stored in the first storage 110 as first digital biomarker data.

The moving object Om displayed on the display 250 moves in a specific direction along a preset path at a preset speed. Gaze information includes at least one of accuracy information that is obtained on the basis of the movement distance of user's eyes and the movement distance of the moving object Om, latency information that is obtained on the basis of the point in time at which the moving object Om starts to move and the point in time at which the user's eyes start to move, and speed information related to the movement speed of the user's eyes.

A stroop test is exemplified in FIG. 5B as cognitive function test information that is provided to analyze cognitive functions. Cognitive function test information is test information for classifying the stages of dementia of a user (e.g., a normal cognitive group, a low cognitive group, etc.), and answer data to the cognitive function test information are used to determine the conditions of the cognitive functions of the user.

The stroop test is performed through the first application in the user terminal 200, whereby a test image shown in FIG. 4B is displayed on the display 250. Four ‘3’s are displayed in a first region A1 and four numerals ‘1, 2, 3, and 4’ are displayed in a second region A2, and in this state, a user selects one numeral, which is the same as the numerals shown in the first region A1, from the numerals shown in the second region A2. When one of the numerals shown in the second region A2 is selected through the user interface 240 in accordance with an instruction by a user, the numeral is transmitted to the agent device 100 and stored in the first storage as answer data.

In this case, test information that the user answers is standard test information and includes all of items for analyzing cognitive functions, and the standard test information is provided every three months and used to analyze the cognitive functions of the user.

Meanwhile, the stroop test is exemplified as cognitive function test information in FIG. 5B, but the present disclosure is not limited thereto, and it is possible to use one or more tests of various tests, such as an arithmetic ability test, a memory test, and a gaze test, as cognitive function test information.

A result of analyzing user's gaze information and answer data is exemplified in FIG. 5C. The analyzed user's cognitive functions are shown in top percentage (%) for each field, and training content for training abilities having percentage lower than a preset reference of language ability, ability to understand space and time, judgment, abstract thinking power, arithmetic ability, and memory is transmitted from the agent device 100 to the user terminal 200. Assuming that a reference is as 50% in FIG. 5C, training content for training arithmetic ability in top percentage of 56% and memory in top percentage of 60% is transmitted from the agent device 100 to the user terminal 200.

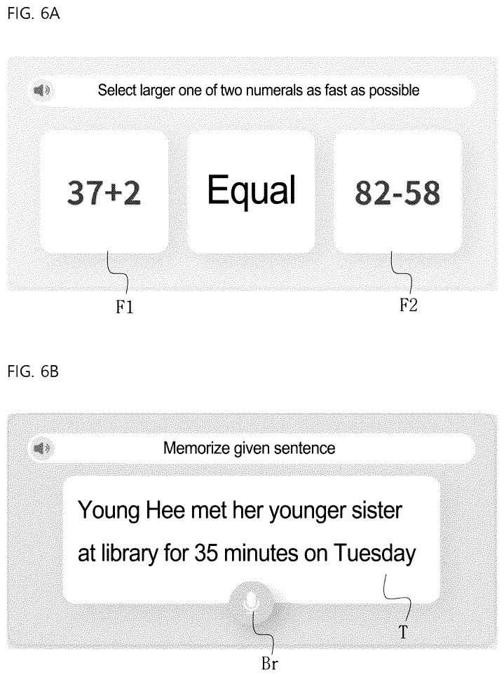

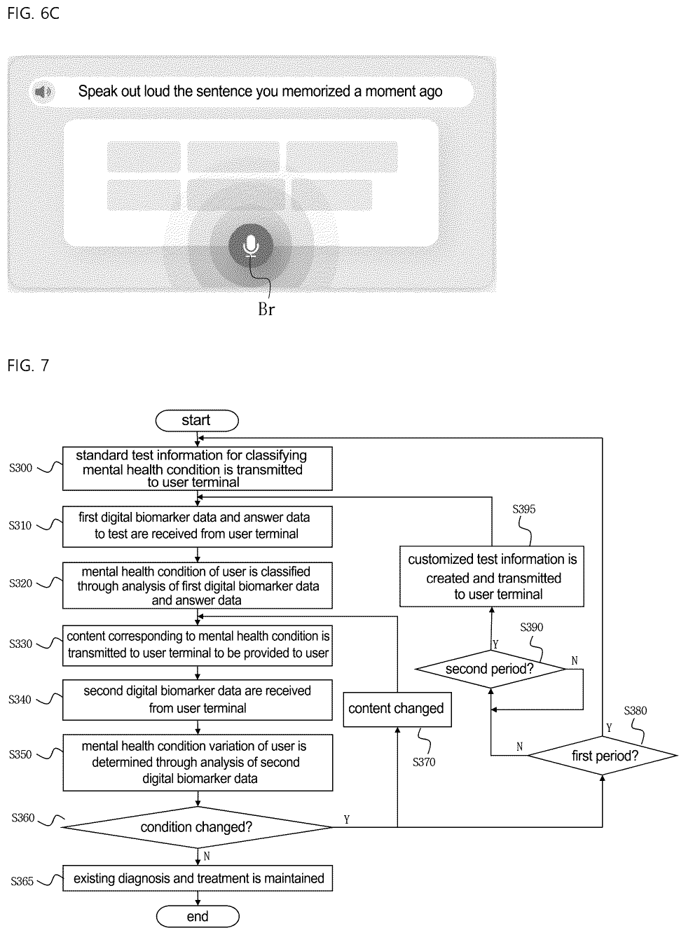

FIGS. 6A to 6C are views illustrating a method of training cognitive functions through the second application in accordance with a second embodiment of the present disclosure. FIG. 6A exemplifies training content for training arithmetic ability and FIGS. 6B and 6C exemplify training content for training memory. The training content for training cognitive functions is not limited thereto, and at least one test of an arithmetic ability test, a memory test, and a gaze test may be used as content for cognitive function training.

Referring to FIG. 6A, when training content for training arithmetic ability is transmitted from the agent device 100 to the user terminal 200 and is executed, an image related to arithmetic ability is displayed on the display 250 through the second application. When any one of a formula F1 and a formula F2 is selected in accordance with an instruction by a user that is input through the user interface 240, response information is transmitted from the user terminal 200 to the agent device 100 and stored as second digital biomarker data in the first storage 110.

In FIG. 6B, a sentence T that is training content for training memory is created in accordance with 5W1H and is then transmitted from the agent device 100 and stored in the second storage 210, and the second processor 220 controls the display 250 such that the sentence T stored in the second storage 210 is provided to a user through the first application. Alternatively, when several words that are different parts of speech and have different meanings are stored in the second storage 210, the second processor 220 creates a sentence T following 5W1H by combining some of the words stored in the second storage 210 and performs control such that the sentence T is displayed through the display 250.

Referring to FIG. 6C, a record button Br is touched, the second processor 220 performs control such that external voices are recorded through the microphone 270. Alternatively, when a preset time passes from the point in time at which a second image including the sentence T was displayed, the second processor 220 activates touch input for the record button Br such that external voices are recorded through the microphone 270. When the user speaks the sentence T with the record button Br activated, the user's voice is recorded through the microphone 270, and is then transmitted to the agent device 100 and stored as voice-related information in the first storage 110.

The first processor 120 determines cognitive function condition variation by analyzing answer information or voice-related information stored in the first storage 110. For example, when it is determined that the percentages of arithmetic ability and memory have been changed as the result of analysis, the first processor 120 performs control such that new training content suitable for the changed conditions is provided through the second application. When the user receives and executes the new training content, the execution result is transmitted to and analyzed in the agent device 100. While this process is repeated, the first processor 120 detects abnormality by continuously checking the execution result by the user. When homeostasis becomes a preset critical level, the first processor 120 detects that there is abnormality, and creates cognitive function test information about arithmetic ability or memory having the abnormality detected, and provides the cognitive function test information to the user in accordance with the period of one month. The user answers the cognitive function test information and the answer data are transmitted back to the agent device 100 and stored in the first storage 110.

FIG. 7 is a flowchart illustrating a content providing method for performing adaptable diagnosis and treatment for mental health according to all embodiments of the present disclosure.

According to FIG. 7, test information for classifying mental health conditions is transmitted to the user terminal 200 (S300). That is, the agent device 100 transmits standard test information to the user terminal 200 in accordance with a preset first period such that a standard test for classifying mental health conditions is provided through a first application installed in the user terminal 200.

First digital biomarker data and answer data to the test are received from the user terminal 200 (S310). That is, first digital biomarker data are obtained through the first application installed in the user terminal 200 and the answer to the test provided through the first application is input to the user terminal 200. The agent device 100 receives the first digital biomarker data and the answer data that are transmitted from the user terminal 200. The first digital biomarker data include at least one or more items of information of Heart Rate Variability (HRV) information of a user, gaze information of a user, voice-related information of a user, and response time information of a user. In this case, the HRV information is extracted in a non-contact type by estimating a heart response by analyzing a color change from a real-time face image. The gaze information of a user is extracted by analyzing variation of the user's gaze from an image including the user's eyes taken when the user's gaze follows a moving object.

The mental health condition of the user is classified by analyzing the first digital biomarker data and the answer data (S320). A mental health condition means a condition in which the person can always independently and autonomously handle normal life, has resistance against diseases, and can do smooth individual and social life, that is, a mentally mature condition. Mental health conditions related to mental disorders or cognitive functions are included in the mental health condition stated in the present disclosure.

The agent device 100 determines the mental health condition of the user who uses the user terminal 200 by analyzing the answer data together with the first digital biomarker data. That is, as shown in FIGS. 3A to 3C described above, mental disorders are diagnosed by analyzing HRV data of the user and answer data about mental disorder question information. Alternatively, as exemplified in FIGS. 5A to 5C, cognitive functions are diagnosed by analyzing gaze information of the user and cognitive function classification information.

Content for corresponding to the mental health condition is transmitted to the user terminal 200 to be provided to the user (S330). That is, when a mental disorder is diagnosed, Self-affirmation and Acceptance and Commitment Therapy (ACT) content for treating mental disorders is transmitted to the user terminal 200 and provided to the user through the second application, as exemplified in FIGS. 4A to 4C. Alternatively, when cognitive function conditions are diagnosed, arithmetic ability and memory training content for training cognitive abilities lower than a preset reference is transmitted to the user terminal 200 and provided to the user through the second application, as exemplified in FIGS. 6A to 6C.

Second digital biomarker data are received from the user terminal 200 (S340). That is, the second application is executed in the user terminal 200, whereby the treatment content transmitted from the agent content 100 is provided to the user. Further, the voice of the user reading the provided treatment content is created as second digital biomarker data in the user terminal 200 and transmitted to the agent device 100. Alternatively, the second application is executed in the user terminal 200, whereby the training content transmitted from the agent content 100 is provided to the user. Further, data corresponding to the result of executing the provided training content by the user are created as second digital biomarker data in the user terminal 200 and transmitted to the agent device 100. The second digital biomarker data is any one of voice-related information of the user and response information about at least one test of an arithmetic ability test, a memory test, and a gaze test.

Mental health condition variation of the user is determined by analyzing the second digital biomarker data (S350), and when there is no condition variation (S360-N), the existing diagnosis and treatment method is maintained (S365). When it is determined that mental health condition variation exists (S360-Y), the existing content is changed (S370) and customized test information to be provided to the user from the agent device 100 is created and transmitted to the user terminal 200 (S395).

Describing in more detail, the agent device 200 determines mental health condition variation in accordance with whether homeostasis becomes a critical level or higher by analyzing the second digital biomarker data. When it is determined that mental health condition variation exists (S360-Y), the existing content that is provided to the user is changed in the agent device 100 (S370) and the steps S330 to S350 are repeated. That is, the content changed in the agent device 100 is transmitted to the user terminal 200 to be provided to the user, second digital biomarker data corresponding to the changed content are received from the user terminal 200, and the agent device 100 determines mental health condition variation of the user by analyzing the second digital biomarker data.

Further, when it is determined that mental health condition variation exists (S360-N) and when the first period is not reached (S380-N) and the second period set shorter than the first period is reached (S390-Y), customized test information excluding items having constant homeostasis in the existing test information is created and transmitted to the user terminal 200 (S395) and the steps S310 to S350 are repeated. That is, customized test information excluding items having constant homeostasis in the existing test information is created and transmitted to the user terminal in accordance with the second period set shorter than the first period. In this case, the standard test information and the customized test information is any one item of information of mental disorder question information that is provided to analyze the mental disorder of the user and cognitive function test information that is provided to classify the stage of dementia of the user.

While steps in which new content is transmitted to the user terminal 200 in accordance with a determination result and new second digital biomarker data are obtained are periodically performed, customized test information is also continuously updated. That is, customized test information excluding items having constant homeostasis is repeatedly created in accordance with the second period and transmitted to the user terminal. Accordingly, a user takes tests only for the items having condition variation, so it is possible to reduce time for tests and the fatigue of a user responding to tests.

In addition, the agent device 100 can receive log data of each of the first application and the second application from the user terminal 200. Further, the agent device 100 determines whether any one of the first application and the second application has reached a preset number of times or a preset use time with reference to the log data. When there is an application that has not reached the preset number of times or the preset use time in the first application and the second application, alarm information about use of the application is created and transmitted to the user terminal 200. Accordingly, the user can periodically test his/her mental health condition through the first application and can be periodically provided with content suitable for his/her mental health condition through the second application.

What is claimed is:

1. A content providing method for performing adaptable diagnosis and treatment for mental health on the basis of digital biomarker data through at least one processor of a device, the method comprising:
a step in which first digital biomarker data obtained through a first application installed in a user terminal are received from the user terminal;
a step in which a mental health condition of a user who uses the user terminal is classified through analysis of the first digital biomarker data;
a step in which content corresponding to the mental health condition is transmitted to the user terminal to be provided to the user through a second application installed in the user terminal;
a step in which second digital biomarker data obtained through the second application are received from the user terminal;
a step in which mental health condition variation of the user who uses the user terminal is determined through analysis of the second digital biomarker data; and
a step in which the content is changed and transmitted to the user terminal in accordance with the mental health condition variation.

2. The method of claim 1, further comprising a step in which when homeostasis, which is property of a human body trying to maintain an internal environment at a constant level becomes a critical level or higher, it is determined that there is mental health condition variation and customized test information excluding items having constant homeostasis in standard test information transmitted to the user terminal in accordance with a preset first period is created and transmitted to the user terminal in accordance with a second period set shorter than the first period.

3. The method of claim 1, further comprising:
a step in which standard test information is transmitted to the user terminal in accordance with a preset first period such that a standard test for classifying the mental health condition is provided through the first application installed in the user terminal; and
a step in which answer data to the standard test are obtained through the first application and received from the user terminal,
wherein the step in which the mental health condition of the user who uses the user terminal is classified is a step in which the answer data are analyzed together with the first digital biomarker data, whereby the mental health condition of the user who uses the user terminal is classified.

4. The method of claim 3, wherein the step in which mental health condition variation is determined is a step of determining that there is the mental health condition variation when homeostasis becomes a critical level or higher by analyzing the second digital biomarker data.

5. The method of claim 4, further comprising a step in which when it is determined that there is the mental health condition variation, customized test information excluding items having constant homeostasis in the standard test information is created and transmitted to the user terminal.

6. The method of claim 5, further comprising a step in which when it is determined that the mental health condition variation exists, the customized test information excluding items having constant homeostasis is repeatedly created and transmitted to the user terminal at every second period set shorter than the first period.

7. The method of claim 5, wherein the standard test information and the customized test information is any one of mental disorder question information that is provided to analyze a mental disorder of the user and cognitive function test information that is provided to classify a stage of dementia of the user.

8. The method of claim 1, wherein the first digital biomarker data include at least one or more items of information of Heart Rate Variability (HRV) information of the user, gaze information of the user, voice-related information of the user, and response time information of the user.

9. The method of claim 8, wherein the heart rate variability information of the user is extracted in a non-contact type by estimating a heart response by analyzing a color change from a real-time face image.

10. The method of claim 8, wherein the gaze information of the user is extracted by analyzing variation of a user's gaze from an image including user's eyes taken when the user's gaze follows a moving object.

11. The method of claim 1, wherein the content is any one of treatment content for treating a mental disorder of the user and a training content for training a cognitive function of the user.

12. The method of claim 1, wherein the second digital biomarker data is any one of voice-related information of the user and response information about at least one test of an arithmetic ability test, a memory test, and a gaze test.

13. The method of claim 1, further comprising:
receiving log data of each of the first application and the second application;
determining whether any one application of the first application and the second application has reached any one value of a preset number of times of use and a preset use time with reference to the log data; and
creating alarm information about use of an application that has not reached any one value of the preset number of times and the preset use time and transmitting the alarm information to the user terminal.

14. A computer program stored in a computer-readable storage medium, the computer program performs steps for performing adaptable diagnosis and treatment for mental health on the basis of digital biomarker data when the computer program is executed by at least one process of a device, wherein the steps comprise:
a step in which first digital biomarker data obtained through a first application installed in a user terminal are received from the user terminal;
a step in which a mental health condition of a user who uses the user terminal is classified through analysis of the first digital biomarker data;
a step in which content corresponding to the mental health condition is transmitted to the user terminal to be provided to the user through a second application installed in the user terminal;
a step in which second digital biomarker data obtained through the second application are received from the user terminal;
a step in which mental health condition variation of the user who uses the user terminal is determined through analysis of the second digital biomarker data; and
a step in which the content is changed and transmitted to the user terminal in accordance with the mental health condition variation.

15. A mental health management system that provides content for performing adaptable diagnosis and treatment for mental health on the basis of digital biomarker data, the system comprising:
a user terminal configured to obtain first digital biomarker data by executing a first application and obtain second digital biomarker data by executing a second application; and
an agent device configured to classify a mental health condition of a user who uses the user terminal by analyzing the first digital biomarker data transmitted from the user terminal, transmit content corresponding to the mental health condition to the user terminal, and determine mental health condition variation of the user who uses the user terminal by analyzing the second digital biomarker transmitted from the user terminal,
wherein the agent device changes and transmits the content to the user terminal in accordance with the mental health condition variation.
